(19) United States
(12) Patent Application Publication
(10) Pub. No.: US 2012/0231464 A1
(43) Pub. Date: Sep. 13, 2012

(54) Heatable Droplet Device

(75) Inventors: Chih-Sheng Yu, Hsinchu (TW); Fan-Gang Tseng, Hsinchu City (TW)

(73) Assignee: Instrument Technology Research Center, National Applied Research Laboratories

(21) Appl. No.: 13/044,658

(22) Filed: Mar. 10, 2011

(51) Int. Cl. C12Q 1/68; C12M 1/34

(52) U.S. Cl. 435/6.12; 435/287.2

(57) ABSTRACT

A heatable droplet device is used to embody real-time detection by means of the device's temperature control and surface treated and trimmed. A temperature causing internal stability disturbed is immediately detected with a designed sensor while affecting a specific area.

### BACKGROUND OF THE INVENTION

1. Field of the Invention

The present invention is a heatable droplet device which is widely applied to the inspection equipment requiring temperatures rapidly controlled such as real-time quantitative PCR equipment and RNA reverse transcription PCR (RT-PCR) equipment for better reaction efficiency and substantial real-time quantitative detection.

2. Description of the Prior Art

In the wake of rapidly progressive Micro-Electro-Mechanical System (MEMS) and biomedicine technologies, there have been many technologies applied to medical care and triggering research and development of small, personal, nano-level, customized, and wireless medical appliances as well as green medical appliances for energy saving and carbon reduction consequently.

As one technology to minimize biochips, Micro-Electro-Mechanical System (MEMS) which evolves into the Bio-MEMS technology with biomedicine cooperatively applied is intended for developing small portable instruments for fast detection. In the biomedicine field, temperature is a common factor affecting catalysis in a biochemical reaction of PCR Technology, RT-PCR, Digest, etc.

The prior art to develop a micro-header with the MEMS process & technology [Journal of Thermal Sciences, 46, 580-588, 2007] is characteristic of depositing two metals, Au and Ti, on a glass substrate as a heater to which a constant voltage is given for a heat source substantially generated. Accordingly, Power (P) is given by Eq. (1):

P=i²R (1)

i: Current; R: Metal resistance

Polymerase Chain Reaction (PCR) Technology

In 1985, Kary Mullis who invented Polymerase Chain Reaction (PCR) had the honor of the Nobel Prize in Chemistry and patents [U.S. Pat. No. 4,683,195] [U.S. Pat. No. 4,683,202]. There are three steps in PCR: (1) Denature: Ascend a temperature to 94° C. and break the double strand in a DNA template. (2) Annealing: Descend a temperature to 50-65° C. and introduce a pair of primers into a double-strand DNA molecule for detecting and linking a complementary base sequence. (3) Extension: Ascend a temperature to 65-75° C. for activating, polymerizing, and linking 3′ of the primer, and follow the base sequence on the template to catch ambient corresponding dNTP for development of new nucleic acid molecule chains; two polymerases comply with the template and face-to-face grow to become nucleic acid molecule chains.

Conventionally, a fast heating/cooling module is used to heat metal plates inside a cavity of PCR equipment and conduct thermal energy from heated plates to plastic tubes (minimum volume: 15 ul) on metal plates for three types of thermal cycles (temperatures: 65, 95, and 75). Simultaneously, a bio-signal is amplified by 2ⁿ (n=1 for one cycle) to maximize a very small biomedical signal with n increased to 25 or 30. In recent years, a real-time detection technology has been embodied with a fluorescent detection system integrated.

Recently, a PCR process with two-stage temperatures for the overall reaction time substantially reduced is to integrate temperatures in annealing/extension and remain the original design for denature so that there are two temperatures only in the whole reaction for completion of a PCR process. However, the overall time is still increased due to a heating/cooling procedure in a conventional heating system.

Except for progress of reagents, micro-heaters developed with the MEMS technology and applied to the biomedicine field have their advantages to rapidly ascend or descend temperatures and reduce a reagent's volume.

Among all PCR technologies developed recently and gradually applied to real-time detection, the optical detection technology is mostly applicable. As one fluorochrome commonly used in a fluorescent real-time detection system, SYBR Green is characteristic of being embedded in double-strand DNA molecules and generates high-intensity fluorescence with the quantity of double-strand DNA molecules increased in a PCR process. Generally, precise optical components, excitation light source (such as laser), and precise & accurate optical lens unit are necessary to conventional equipment.

In 1958, Palecek found DNA presented redox reactions on electrochemical electrodes. Consequently, the DNA-related electrochemical detection is employed. For effective real-time detection of an amplified DNA, a reagent such as methyl blue which reacts with DNA is added and embedded into double helix DNA molecules so that current signals occurred in a reaction are reduced. Accordingly, the real-time detection of one PCR process can be materialized in this way. A device with the electrochemical detection and the DNA immobilization technology employed ([U.S. Pat. No. 7,135,294] and [U.S. Pat. No. 7,393,644]) allows DNA to be fixed on the surface of one substrate and gives reagents to DNA molecules in a PCR process for measurements of impedance signals.

In general, there are many ways available in measurement of DNA such as sensors on an electrode's surface for detection of DNA due to a nanostructure with a highly contactable surface area and a nano surface electrode capable of directly measuring or favorably detecting DNA.

Manufactured in a MEMS process, the present invention is a micro-heater available in a biochemical process for not only thermal energy supplied to biochemical detection but also detective elements on one chip's surface as media for real-time detection in a biochemical reaction. In virtue of design of the chip, a biological molecule is driven to a specific direction and detected in a specific area by detectors therein.

Due to the Free Convection effect of thermodynamics caused by changes in temperatures and densities, an unsteady circulation from changeable temperatures leads to a velocity field in one liquid changed. A flow field will be automatically generated in virtue of changes in buoyant particles and densities inside a heated liquid. Accordingly, liquid molecules are driven to pass some specific areas such as electrode and optical detection area. With manufactured micro-electrodes heated, the equipment of the present invention is capable of driving both a temperature on a droplet's central bottom up to 95° C. and heated molecules' buoyancy by which molecules are driven upward along a path subjected to a droplet's external geometric shape and thus move toward a droplet's periphery while arriving at the droplet's top. On the other hand, biological molecules arriving at a droplet's heated bottom are driven upward. In this way, the thermal circulation of a polymerase chain reaction is completed.

In view of a critical issue for liquids evaporated during a heating process, mineral oil is usually taken as one liquid to prevent evaporation in a PCR process. The device of the present invention is designed to store two types of liquids on its surface and define an area for their storage with both photo resist SU-8 and standard photolithography for mineral oil preventing reagents in a PCR process from evaporation during a heating process.

### Temperature Control Method (First Temperature Fixed; Second or Other Temperatures Adjusted)

To accurately control a temperature, a feedback voltage is used to monitor thermal power realistically produced, i.e., the consumable power (P) based on a required result is fixed with a manufactured confirmable metal resistance (R) and a constantly controlled current (from Eq. (1)) for a precise control of a temperature in a biochemical experiment. As such, a temperature control mechanism to adjust and monitor a heater's status is developed by constantly cooling a substrate. On the other hand, a determinand driven to pass a detector's surface by turbulence induced in a flow field allows signals to be extracted, for instance, signals by means of the function of “Plus” given to different temperatures detected under control of software.

For a temperature automatically controlled and loaded, a chip automatically loaded by a designed mechanism precisely contacts with a securely fixed probe card without man-made mistakes or contaminations. As such, signals from a chip can be transmitted with a probe card and metal wires.

In view of a precisely controlled temperature in biomedicine, a micro-heater is advantageous to fast response, low energy consumption, and rapid temperature changes and applied to various fields such as RT-PCR (reverse transcription), Real-Time PCR, and Digest and further some related industries such as biochemistry and agriculture significantly with the polymerase chain reaction proposed. In contrast to a conventional temperature-based PCR instrument with necessary response time consumed in a biological specimen as well as a PCR process depending on an instrument's stabilized temperature, a device sensitive to micro volume and responding quickly is presented here for real-time detection.

### Real-Time Detection

### Electrochemistry Principle

Based on a redox mechanism of electrochemistry to detect a specimen, the present invention is capable of driving liquid molecules under different temperatures to circularly pass a detector's surface for real-time monitoring of a biological specimen. Furthermore, a specific detection is available with extra surface areas built on a detector's nanostructure as well as a trimmed surface. In addition, the nanostructure on a detector is favorable to changes in liquid molecules' flowing speeds and adjustment of a flow field inside a liquid.

### Fluorescent Detection System

The real-time detection system of the present invention belongs to an optical detection mode partially because semicircular droplets developed as a lens in one reaction focus weak light signals on a detector's surface.

Despite conventional PCR requiring rapid heating/cooling which consumes much power, bulk biological reagents used in a reaction, and a fluorescent microscopy unit increasing the volume of one instrument system for a fluorescent test, the present invention based on the mature and extensively used PCR technology proposes a novel technique applied to the conventional instrument system due to promotion of an energy concept: (1) Reduce power consumption of an instrument system for energy saved effectively; (2) Reduce the volume of biological reagents with a novel minimized PCR chip for detection of a small volume and usage of small biological reagents in view of lots of rare biological specimens unavailable and expensive; (3) Minimized system for material saving and no resource wasted due to a conventional instrument system based on the optical detection causing a massive instrument system.

In consideration of both some defects derived from a real-time quantitative PCR temperature control device based on the prior art and a heatable droplet device extensively applied to fast temperature-controlled detection equipment such as real-time quantitative PCR equipment and RNA reverse transcription PCR (RT-PCR) equipment for better reaction efficiency and substantial real-time quantitative detection, the inventor successfully developed the heatable droplet device after making extraordinarily painstaking efforts and research in many years.

### SUMMARY OF THE INVENTION

The present invention is a heatable droplet device.

The object of the present invention is to use the heatable droplet device for a temperature immediately adjusted and controlled in one reaction.

The further object of the present invention is intended for optical or electrochemical real-time detection of a PCR process completed with the heatable droplet device.

The present invention is demonstrated and interpreted but not restricted by the following embodiments.

These features and advantages of the present invention will be fully understood and appreciated from the following detailed description of the accompanying Drawings.

### BRIEF DESCRIPTION OF THE DRAWINGS

FIG. 1 is the schematic diagram of a heatable droplet device with an electrochemical detection device integrated for real-time detection.

FIG. 2 is the curve for distributed temperatures on a heated electrode.

FIG. 3 is the temperature curve.

FIG. 4 is the schematic diagram of a heatable droplet device with an optical detection device integrated for real-time detection.

FIG. 5 is the curve for real-time measurements of extracted signals.

FIG. 6 is the schematic diagram for changes in the flow field caused by a nanostructure.

FIG. 7 is the schematic diagram for carbon nano tubes on a detective electrode's surface.

FIG. 8 is the curve for test results in the detective area.

FIG. 9 is the schematic diagram for a chip.

### DETAILED DESCRIPTION OF THE PREFERRED EMBODIMENT

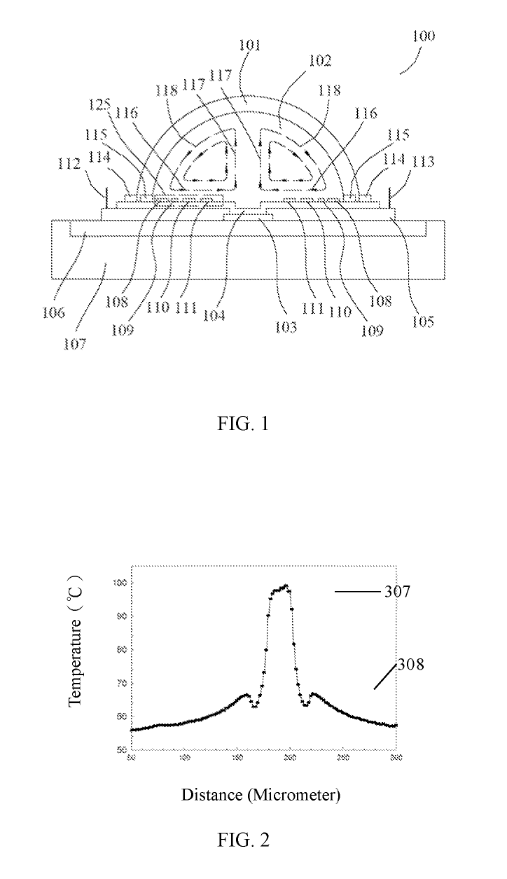

Referring to FIG. 1 which displays details of the present invention of a heatable droplet device for reaction equipment 100 comprising a first liquid 101, a second liquid 102, a heater 103, a protection layer 104, an outer lead 105, a substrate 106, a cooling device 107, a first unit of sensors 108, a second unit of sensors 109, a third unit of sensors 110, a fourth unit of sensors 111, a first signal source 112, a second signal source 113, a first ring layer 114, a second ring layer 115, a first circle line 116, a second circle line 117, and a third circle line 118.

Fabricated with MEMS, an electrode has its central area heated with the metal heater 103 manufactured in platinum and the outer leads 105 (metal leads, for instance, aluminum herein). Based on the principle of heating a resistor, the heater 103 forms a heat resource and thus generates circular traces comprising the first circle line 116, the second circle line 117, and the third circle line 118 as a current or a voltage is introduced to the outer leads 105. Next, a real-time response signal is detected with the first unit of sensors 108, the second unit of sensors 109, the third unit of sensors 110, and the fourth unit of sensors 111 inside a reaction sensing area 125.

For effectively direct detection, a sensing area is designed in the first circle line 116 which dominates annealing and extension for PCR. In the case of heating, a specific temperature reached with the heater 103 will propagate along traces like the first circle line 116, the second circle line 117, and the third circle line 118 and are detected immediately with an electrode unit inside the reaction sensing area 125.

Referring to FIG. 2 which displays the curve for distributed temperatures completed with a voltage (3 volt for 30 μa) from the heater 103, the first signal source 112, the second signal source 113, and the outer lead 105 to stabilize the cooling device 107 and measured with an infrared thermometer wherein a stable first temperature 307 (95) and a second temperature (60) is available in the heater 103 and the cooling device 107, respectively.

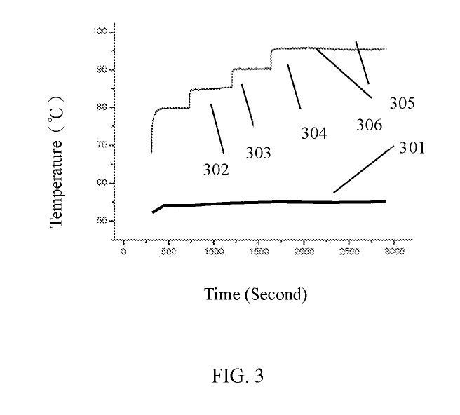

Referring to FIG. 3 which displays fast ascendant and descendent temperatures wherein Curve 301 represents temperatures necessary to cooling the substrate 107 and Curves 302-305 represent various temperatures from the heater 103 with different voltages or currents supplied to 112 and 113. In this way, the temperate-related direct results for heating rate 306 and a fast stable Curve 305 are obtained with signals given.

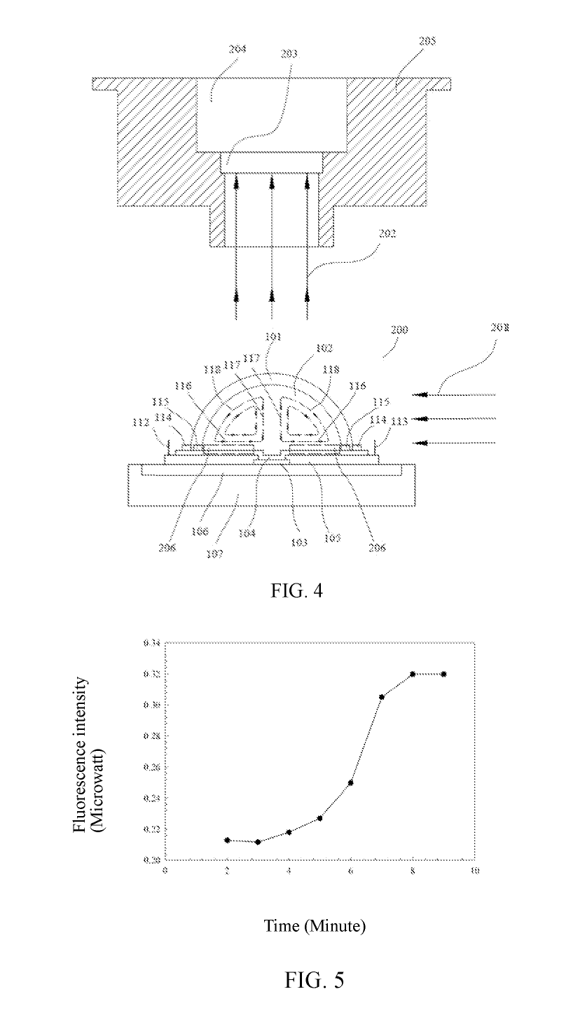

Referring to FIG. 4 which displays reaction equipment 200 comprising a first liquid 101, a second liquid 102, a heater 103, a protection layer 104, an outer lead 105, a substrate 106, a cooling device 107, a first unit of sensors 108, a second unit of sensors 109, a third unit of sensors 110, a fourth unit of sensors 111, a first signal source 112, a second signal source 113, a first ring layer 114, a second ring layer 115, a first circle line 116, a second circle line 117, a third circle line 118, a first light source 201, a second light source 202, a first barrier layer 203, a signal receiver 204, and a support 205. Due to the first light source projected on the surface of the second liquid 102, the first light source 202 (fluorescent signal) generated by amplified biological molecules can be extracted through 203 and detected by the signal receiver 204, as shown in FIG. 4. To control a flow rate inside the second liquid 102 and reach required time as well as reactions, some liquids should be proportionally added into the second liquid 102 for increased coefficients of viscosity (i.e., the second liquid 102 with different coefficients of viscosity) and control of various flow rates. The methods for stimulating and detecting laser are divided into lateral stimulation and top detection or stimulation and detection from a coaxial light source.

### Embodiment 1

### Two-Stage Temperature Control for a Biochemical Reaction

The present invention of a heatable droplet device is embodied with heat dissipation of a micro-scale characteristic of rapid heating and cooling. The two-stage temperature control is used to reach amplified PCR. Firstly, a temperature on the cooling device 107 should be an annealing temperature of 65; for amplified PCR under timing control, this temperature is increased to a denaturing temperature of 95 with the electrode heater 103. This method avoids thermal loss during a heating or cooling process and contributes to a real-time detection with both detection and heating (cooling) simultaneously completed.

Subjected to the electrode heater 103, a three-stage temperature control for 95, 65, and is materialized.

### Embodiment 2

### Electrochemical Mechanism for Measurements of PCR Products

Electrical signals for real-time detection of PCR products: Referring to FIG. 9 which displays the schematic diagram of a chip manufactured in the MEMS process and comprising a first unit of sensors (work electrode) 108, a second unit of sensors (reference electrode) 109, a third unit of sensors (counter electrode) 110, and a fourth unit of sensors (work electrode) 111. While passing an electrode's surface in this system, fluid molecules affected and driven by a temperature field are detected with measured signals (e.g., current) increased or decreased. Accordingly, an electrode on the surface of the heatable droplet device for the present invention contributes to not only the detection sensitivity but also changes in the flow field by means of its nanostructure. A design for an electrode can be either a symmetric or a sandwich structure.

### Embodiment 3

### Optical Mechanism for Measurements of PCR Products

From a droplet, PCR products stimulated by laser can be immediately detected with an optical detection system. In virtue of existing liquid droplets with a feature of focusing light, fluorescent signals generated will be transmitted to a detector, CCD or PMT, for signals effectively amplified as shown in FIG. 5 which displays results stimulated by a lateral light source and detected on the top of liquids that present reactions from DNA in 2 minutes and complete the whole reaction in 10 minutes.

### Embodiment 4

### Micro/Nano Surface Electrode

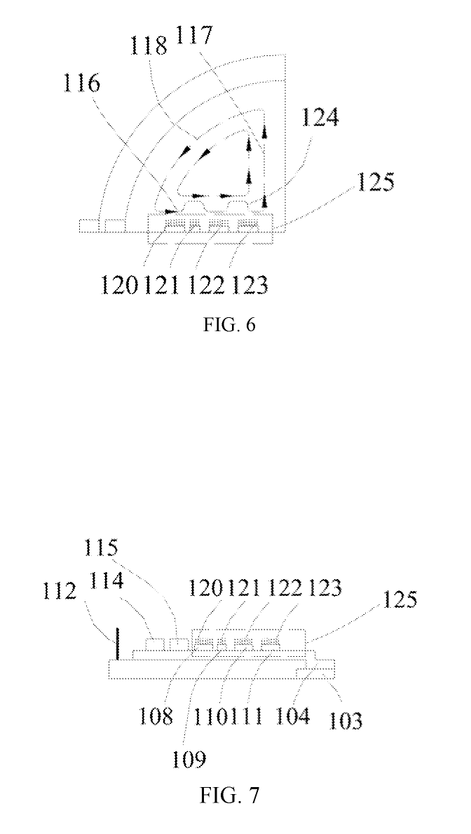

An electrode used in the present invention of a heatable droplet device is also a micro/nano surface electrode (FIG. 7). Referring to FIG. 6 and FIG. 7 which display a first unit of sensing growths 120, a second unit of sensing growths 121, a third unit of sensing growths 122, and a fourth unit of sensing growths 123 accommodated in the sensing area 125 contribute to an electrode herein detecting a determinand driven by 116, facilitate movement of a flow field for development of a migration path (such as 124), change a flow field (FIG. 6) and increase sensitivity. In this regard, the first unit of sensing growths 120, the second unit of sensing growths 121, the third unit of sensing growths 122, and the fourth unit of sensing growths 123 trimmed chemically or physically contribute to a specific reaction.

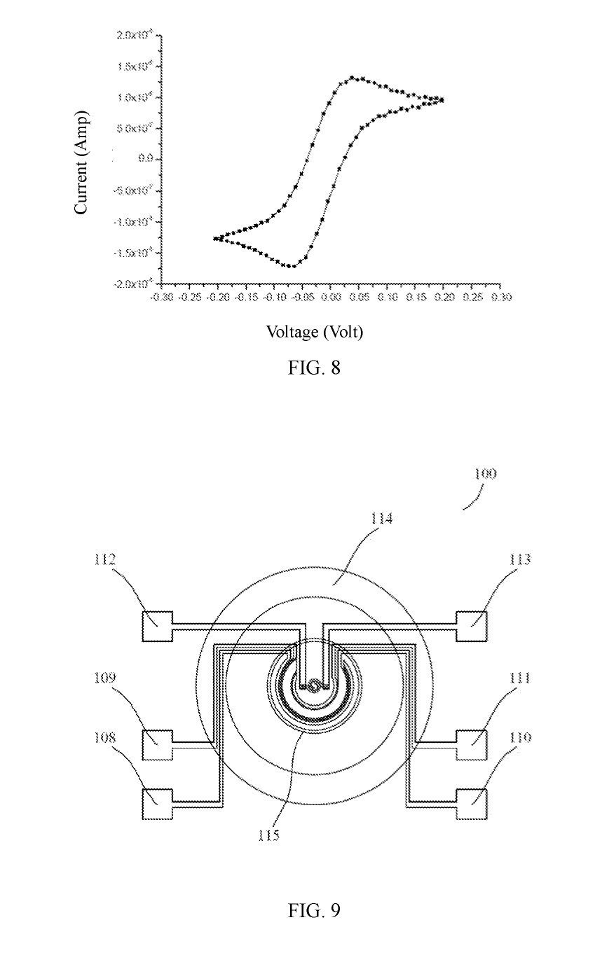

For the purpose of verifying features of the sensing area 125, those features should be measured in accordance with the electrochemical principle. As shown in FIG. 8, the measured results are outcomes with voltages, which are supplied to the first unit of sensors 108 and the third unit of sensors 110 inside the sensing area 125, switched between positive and negative.

### Embodiment 5

### RNA Reverse Transcription PCR (RT-PCR)

The present invention of a heatable droplet device is also applied to temperature control equipment inside a RT-PCR instrument for an effective RT-PCR process with temperatures in an instrument rapidly adjusted.

### Embodiment 6

### Application of Enzyme Digeation

The present invention of a heatable droplet device is applied to a temperature control instrument used in a biochemical reaction such as Enzyme Digeation for fast heating/cooling, reduced thermal loss, and enzyme easily decomposing other substances.

The said details relating to the present invention are specific descriptions of feasible embodiments not restricting claims of the present invention; any equivalent embodiment or change which does not depart from the art or the spirit of the present invention, for instance, the heatable droplet device applied to the equipment requiring fast temperature control such as PCR and RNA reverse transcription PCR (RT-PCR), is included in claims herein.

In summary, the present invention featuring not only its method and style categorized to substantial novelty but also said promoted effects in contrast to habitually used devices should sufficiently comply with legal patentability requirements in novelty and inventive steps and be applied for the patent for claims herein approved.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A heatable droplet device, comprising:
A substrate, a heater, composite liquids, sensors, a cooling substrate, and a liquid storage area wherein the composite liquids are restricted on the heater by the liquid storage area and placed on the substrate, which is located at a cooling substrate and drives liquids to circulate, react, and generate signals detected by the sensor immediately in compliance with a difference in temperatures between the heater and the cooling substrate.

2. The heatable droplet device according to claim 1 wherein the sensor can be CCD, PMT, or metal electrode.

3. The heatable droplet device according to claim 1 wherein the sensor can be manufactured in metal or alloy.

4. The heatable droplet device according to claim 1 wherein the reactive liquid covers the sensor.

5. The heatable droplet device according to claim 1 wherein the sensor includes but is not limited to a material with a nanostructure.

6. The sensor in the heatable droplet device according to claim 5 wherein the nanostructure allows its reactive sensitivity to be promoted with its surface trimmed.

7. The heatable droplet device according to claim 1 wherein the circulation rate of the liquid can be adjusted with a nanostructure.

8. A method for real-time detection wherein the method embodies real-time detection by the heatable droplet device according to claim 1 with an optical or electrochemical detection system integrated.

9. The method for real-time detection according to claim 8 wherein the electrical signal can be a change in current and the optical signal can be a fluorescent signal.

10. The method for real-time detection according to claim 8 wherein the liquid has the shape of a droplet or a semicircle lens for fluorescent signals focused on the sensor.

11. The method for real-time detection according to claim 8 wherein the reactive liquid is immediately detected while passing the sensor.

12. The method for real-time detection according to claim 8 wherein the composite liquids comprise reagents and non-evaporated liquids.

13. The method for real-time detection according to claim 8 wherein the method can be applied to PCR, Digest, and RT-PCR.

14. The method for real-time detection according to claim 8 wherein the different temperatures in a reaction can be adjusted and controlled with the heater and the cooling substrate.
